(19) United States
(12) Patent Application Publication
(10) Pub. No.: US 2017/0365974 A1
(43) Pub. Date: Dec. 21, 2017

(54) SPECTRALLY PURE SHORT PULSE LASER

(71) Applicant: LUMINIT LLC, Torrance, CA (US)

(72) Inventors: Russell KURTZ, Torrance, CA (US); Alkan GULSES, Torrance, CA (US)

(21) Appl. No.: 15/182,841

(22) Filed: Jun. 15, 2016

(51) Int. Cl. H01S 3/106; H01S 3/11; G01N 33/00; G01N 21/39; H01S 3/094; H01S 3/16; H01S 3/0941

(52) U.S. Cl. CPC H01S 3/1062; H01S 3/1643; H01S 3/1608; H01S 3/1103; H01S 3/0941; G01N 21/39; H01S 3/094076; G01N 33/0047; G01N 33/004; G01N 2021/396

(57) ABSTRACT

A short-pulse, narrowband, line-selectable and tunable solid-state laser is described. The device requires a pump source, an active solid-state laser medium, an enclosing cavity, mirrors to contain the light, a method of removing the pulse from the cavity, a wavelength selection system, and a laser linewidth narrowing system. One implementation of this is an Er:YAG laser, side pumped by semiconductor lasers in the erbium absorption band near 1475 nm, with an intracavity etalon and a switchable spectral filter. To remove the pulse from the cavity, cavity dumping issues, which assures constant pulse energy and pulse length over a range of repetition rates, in this case from 100 Hz to 20 kHz. Line selection is obtained by use of wavelength filters and fine tuning with an etalon, which also acts as the linewidth narrowing system.

This invention is in the field of optical detection of gaseous compounds, and more particularly the optical detection of pollutants by laser.

To help with the detection of carbon dioxide, methane, and certain gaseous pollutants, a narrow linewidth, tunable and band-selectable, diode-pumped solid-state laser in the wavelength region between 1550 nm and 1650 nm is desired. These lasers must be fieldable and easy to use, and have pulse energy >1 mJ at repetition rate adjustable up to 20 kHz, with pulse length no greater than tens of nanoseconds. The subject matter is an Er:YAG laser, side pumped by semiconductor lasers in the erbium absorption band near 1475 nm, with an intracavity etalon and a switchable spectral filter. The etalon will reduce the laser linewidth to <50 MHz because, unlike traditional Q-switched short pulse lasers, the cavity-dumped apparatus of the subject invention circulates the photon flux through the etalon hundreds of times before sending it out. Cavity dumping also ensures constant pulse energy over a wide range of repetition rates, as low as 100 Hz and as high as 20 kHz. The simple, automated system design ensures environmental ruggedness and ease of use.

### BACKGROUND OF THE INVENTION

The effects of climate change are already becoming severe, and may become disastrous. To prepare, and to avoid climate change problems, it is necessary to monitor the concentration and location of gases that lead to global warming, such as carbon dioxide (CO₂) and methane (CH₄). Currently, there are no portable, accurate tests for these greenhouse gases, although some progress has been made in using differential atmospheric lidar to measure, for example, CH₄ leakage from petroleum processing plants. To help with the detection of these gases, and of other pollutants, a laser of the following characteristics is desirable:
Line selectable to the CO₂ range (1570 nm) and the CH₄ range (1645 nm); tunable over >1 nm within these ranges
Linewidth as narrow as possible (target ˜50 MHz)
Pulsed, >1 mJ/pulse in <10 ns, adjustable repetition rate 1 kHz-20 kHz
Simple, turnkey operation in an environmentally robust system
Less complex than an optical parametric oscillator.

There are several systems under development to address this challenge. There is an optical parametric oscillator under development for detection of CO₂, and a complex Er:YAG laser has been tested for detection of CH₄, but both these systems are designed for use in the laboratory, not the field. In an attempt to make a single laser that can detect several greenhouse gases and other pollutants, Er:YAG lasers have been developed that are line selectable; these are comparatively complex and it is not always easy to select the wavelength band of these non-tunable lasers.

### CONCISE DESCRIPTION OF THE DRAWINGS

FIG. 1 is a schematic of the laser apparatus of the subject invention.

FIG. 2 shows the four subsystems of the apparatus of the subject invention.

FIG. 3 shows the pumping and laser levels of the laser apparatus of the subject invention.

FIG. 4(a+b) shows the laser apparatus of the subject invention using a non-linear crystal in a half wave (a) and a quarter wave (b).

FIG. 5(a+b) shows adjustment of the loss rate for adjusting the pulse rate.

### SUMMARY OF THE INVENTION

To address this need an Er:YAG laser has been developed, side pumped by semiconductor lasers in the Er³⁺ absorption band near 1475 nm, with an intracavity etalon for both linewidth narrowing and tuning within a wavelength band, and with a switchable spectral filter to select the wavelength band itself. The etalon will reduce the laser linewidth to near the uncertainty limit (˜50 MHz) because, unlike traditional Q-switched short pulse lasers, the cavity dumped apparatus of the subject invention circulates the photon flux through the etalon hundreds of times before sending it out. Cavity dumping also ensures constant pulse energy over a wide range of repetition rates, as low as 100 Hz and as high as 20 kHz (previous work indicates 100 kHz is achievable). The simple, automated system design ensures environmental ruggedness and ease of use.

### DETAILED DESCRIPTION OF THE INVENTION

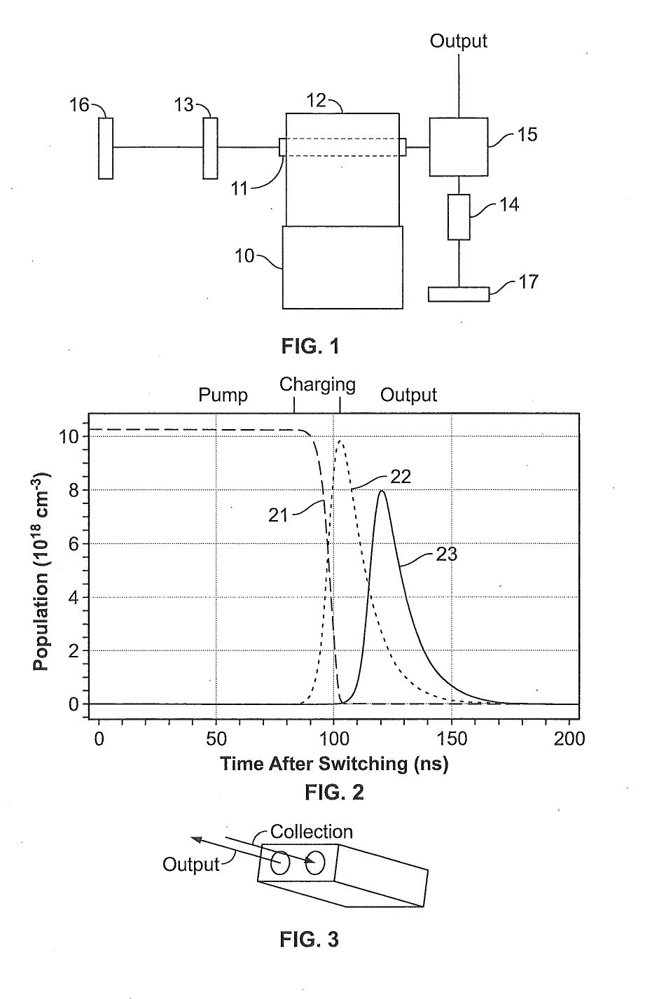

The laser apparatus 30 of the subject invention comprises four subsystems in FIG. 2: pump and power 10, laser 11, spectral 12, and pulse forming 13. Subsystem 10 takes electrical power—generally from a battery but wall-plug power can be used—and first converts it to the forms needed by the system, then uses it to drive semiconductor pump lasers at ˜1.47 μm. This subsystem includes the power to drive the electro-optical modulator (nonlinear crystal) in Subsystem 13, the switchable spectral filter in Subsystem 12, and whatever temperature control is needed. Subsystem 11 is the main laser portion, including the pump cavity, rod, and mirrors. It takes the pump light and converts it into a high coherent beam at the desired wavelength. Subsystem 12, spectral control, includes an etalon and a switchable spectral filter. Subsystem 13 is the pulse forming subsystem.

Of all the laser materials known, the one with the best properties in the wavelength region of interest is the Er³⁺ ion, which can be doped into a number of crystals. Spectroscopic and Judd-Ofelt analysis of these doped crystals, however, indicate that only Er:YAG has the potential to produce laser radiation at the specific wavelengths of interest—near 1570 nm (CO and H₂S absorption) and near 1645 nm (CH₄ and NH₃ absorption).

Er:YAG has two major challenges. First, the laser wavelengths of interest terminate in the ground state of Er³⁺, significantly increasing the lasing threshold and introducing significant temperature effects; this is partially mitigated by careful selection of the semiconductor laser pump wavelength, making Er:YAG a “quasi-three-level” laser. The second challenge is cross-relaxation, in which two ions in the ⁴I_{13/2} level, the upper laser level, interact, pushing one into upper levels with moderate relaxation times, and dropping the other into the ⁴I_{15/2} lower laser level. This is mitigated by using lower concentration of Er³⁺ ions.

Er:YAG is known to produce laser output in the ranges of 1.546 μm, 1.570 μm, 1.645 μm, and 1.618 μm. There are 56 laser wavelengths theoretically possible, owing to seven Stark sublevels in the upper laser level and eight in the lower laser level. In addition, the lower four sublevels of the lower level are in a group with a nearly 350 cm⁻¹ gap before the lowest of the upper four sublevels. These start at 410 cm⁻¹, and thermal population distributions demonstrate that <11.5% of the total population of that level will be in the upper four sublevels. This is critical in quasi-four-level lasers; low thermal population of the terminal laser sublevel enables creation of an inversion at significantly lower pump levels than would be anticipated using standard rate equation laser models. As will be shown below, the lower the sublevel in the upper laser level and the higher the sublevel in the lower laser level, the lower the pump rate needed to reach threshold.

The ground level of Er:YAG, the ⁴I_{15/2}, is also referred to as the Z level; the eight Stark sublevels are labeled Z1-Z8. Likewise, the first excited level, the ⁴I_{13/2}, has sublevels Y1-Y7. The proposed invention will be pumped at ˜1475 nm, from the Z1 sublevel to the Y4 and Y5 sublevels (this value is selected based on previous Er:YAG lasers developed by the proposed Principal Investigator). The initial sublevel contains 27.3% of the total Er³⁺ population at room temperature, and at the start of pumping; as population is pumped out of this sublevel it is replenished, effectively immediately, by thermalization within the sublevels.

The 1570 nm transitions are Y4→Z5 and Y5→Z7. The Y4 and Y5 levels contain 9.11% and 8.31%, respectively, of the total population of the upper laser level, while the Z5 and Z7 levels contain 3.81% and 2.25% of the total population of the lower laser level. The 1645 nm transitions are more likely, partly because the initial Y2 and Y3 levels contain 22.0% and 21.3%, respectively, of the upper laser level population, while the terminal Z7 level contains only 2.25% of the total population in the lower laser level. (All these calculations assume T≈300 K.)

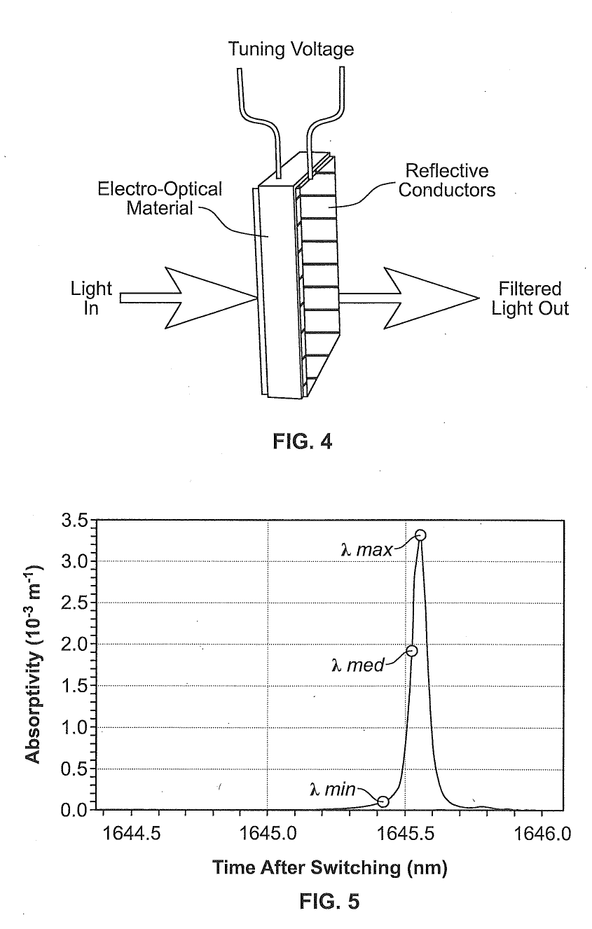

To model the laser operation, we use rate equation analysis. Because of the ion-ion cross-relaxation interaction, the lowest three levels must be considered. (The cross-relaxation actually pumps one ion from the ⁴I_{13/2} level into the ⁴I_{9/2}, which is not included, but it rapidly decays from that level into the ⁴I_{11/2}, which is included.) In these equations, the lower laser level, level 0, is the ground level, the ⁴I_{15/2}; the upper laser level, level 1, is the ⁴I_{13/2}; and the additional excited state, level 2, is the ⁴I_{11/2} level. Levels 1 and 2 are shown in FIG. 4. The rate equations are:

$$\begin{matrix}\frac{\partial {n}_{0}}{\partial t} = - {W}_{01} {n}_{0} + \frac{{n}_{1}}{{τ}_{1}} + {W}_{cr} {n}_{1}^{2} + c σϕ n & ( 0 \text{-} 1 ) \\ \frac{\partial {n}_{1}}{\partial t} = {W}_{01} {n}_{0} + \frac{{n}_{2}}{{τ}_{2}} - {W}_{cr} {n}_{1}^{2} - \frac{{n}_{1}}{{τ}_{1}} - c σϕ n & ( 0 \text{-} 2 ) \\ \frac{\partial {n}_{2}}{\partial t} = {W}_{cr} {n}_{1}^{2} - \frac{{n}_{2}}{{τ}_{2}} & ( 0 \text{-} 3 ) \\ \frac{\partial ϕ}{\partial t} = c σϕ n - \frac{ϕ}{{τ}_{c}} & ( 0 \text{-} 4 )\end{matrix}$$

plus the composition equations

n=f₁ₛn₁−f₀ₛn₀ (0-5)

n₀+n₁+n₂=n_{T}. (0-6)

In eqs. (0-1) through (0-6), nᵢ is the population density of the i^{th} level (i=0, 1, 2); n_{T} is the total population density of all Er³⁺ ions in the crystal; n is the inversion density; fᵢⱼ is the portion of level i population that is in sublevel j; φ is the photon density in the cavity (at the laser wavelength); c is the speed of light; σ is the emission cross-section for the laser transition; W₀₁ is the pump rate from the semiconductor pump laser; W_{cr} is the cross-relaxation rate; and τₖ is the spontaneous lifetime of population in level k (k=1, 2, 3, or c, where c is the cavity lifetime of photons). Numerically solving these equations enables prediction of the output of the laser under any conditions.

As an example, the pump rate needed to reach inversion for the wavelength ranges listed (1570 nm and 1645 nm) as calculated. The relevant fill levels are f₁₂=0.220, f₁₃=0.213, f₁₄=0.0911, f₁₅=0.0831, f₀₄=0.190, f₀₅=0.0381, and f₀₇=0.0225. Thus, to reach threshold for 1570 nm, n₁>0.271 n₀, while threshold is reached for 1645 nm when n₁>0.102 n₀. In other words, it takes 2.7× as much pump intensity to reach threshold for a 1570 nm laser as for a 1645 nm laser. A simple calculation shows that, for 0.25% Er³⁺ doping (the level planned), the required pump rate to reach threshold is ˜8.5×10⁻⁷ μs⁻¹ for lasing at 1645 nm, 2.3×10⁻⁶ μs⁻¹ for 1570 nm.

Most diode-pumped solid-state lasers are end-pumped, to take advantage of the length of the crystal rod. This is less efficient with quasi-four-level lasers. The pump laser has to be nearly the same diameter the entire length of the crystal, and this normally implies a Gaussian profile. On the other hand, the pump rate at a specific location on the crystal is proportional to the intensity of the pump laser, which is much higher at the center of the Gaussian beam. Since an area that is not inverted is, by nature, absorbing (causing loss), the end-pumped beam must waste a large portion of its intensity outside the laser crystal.

Side pumping is more effective for a number of reasons. For one, there is no need for sharp dichroic mirrors; an end-pumped laser would require, in this case, a mirror that passes 1475 nm with almost no loss but reflects almost 100% at 1570 nm, a difficult feat. In addition, end pumping limits the internal cavity layout that can be used to one that enables capture of the entire pump beam, while side pumping places no such limits on the cavity. On the financial side, an end-pumped laser requires a pump laser with a very high quality beam, while a side-pumped laser has virtually no beam quality requirements; the pump laser for a side-pumped system is much less expensive.

Although the effective cross-section is increased from 0.45×10⁻²⁰ cm² to 6.8×10⁻²⁰ cm², based on a 5 mm diameter rod the absorption per pass is still ˜20%. Thus the pumping is nearly uniform. In addition, the design of the cavity ensures that the highest intensity of pump radiation is at the center of the rod, helping optimize the mode shape.

The pump rate can be calculated as

$${W}_{01} = \frac{{σ}_{abs} {I}_{pump}}{{hv}_{abs}} = ( 0.51 {cm}^{2} \text{/} J ) {I}_{pump} , \qquad ( 0 \text{-} 7 )$$

so a pump rate of 0.85 s⁻¹, required to reach inversion for the 1645 nm laser, relates to an average irradiance of 1.7 W/cm²-14 W total pump power for a 5 cm long rod. Pumping above this level puts the majority of power into the laser output, ensuring that the apparatus can reach the desired pulse energy of up to tens of mJ per pulse, at repetition rates up to 20 kHz.

The topic requests a pulse in “tens of ns” and spectral width “as close to transform limited as is practical.” By “transform limited,” what is typically meant is the linewidth is about equal to the inverse of the pulse length; for a 20 ns pulse, for example, Δν=50 MHz is transform limited. This is almost impossible to reach with Q-switched lasers. The methods of narrowing linewidth involve filters, and become much more effective when the photon flux passes through the filters a number of times. In Q-switching, however, the photon flux has little time to pass through the spectral filter. In addition, Q-switching puts the laser in a high-gain mode, so the filter would need to be significantly narrower to reduce the linewidth sufficiently.

In contrast, cavity dumping involves a buildup of the photon flux for a number of cavity round-trips before it is coupled out of the cavity. In this way, we can produce a shorter pulse and still reach the narrow linewidth required.

The absolute limit on the linewidth is the Heisenberg uncertainty principal which, in this case, may be written

$$Δ v Δ t > \frac{1}{2 π} ; Δ v > \frac{1}{2 πΔ t} . \qquad ( 0 \text{-} 8 )$$

In eq. (0-8), Δt is the pulse length and Δν is the spectral linewidth. If, for example, the cavity-dumped pulse length is 5.2 ns, the limitation on the linewidth is Δν>30 MHz—narrow enough to be considered transform limited for a 20 ns pulse.

To achieve this narrow linewidth, it is necessary to add an etalon 15 to the cavity, as shown in FIGS. 1 and 2. The initial cavity design, as shown there, has an optical length 29 cm; the cavity modes, then, are spaced by 520 MHz. To reach the linewidth needed, only one mode may be allowed to oscillate. The etalon 15 (assuming 2.0 cm thickness) has free spectral range of 5 GHz. If the finesse of the etalon is only 2, the effective spectral width of the etalon will be <30 MHz for a cavity-dumped system.

The other purpose for using the etalon 15 is tuning the center line of the laser. This technique has been used to tune Er:YAG over >3 nm. Tuning is accomplished by rotating the etalon, which shifts its center wavelength. This will enable wavelength tuning, for example, from 1567-1573 nm, from 1643-1647 nm, and from 1615-1619 nm.

The apparatus of the subject invention is designed to be a “turnkey” system, with all adjustments made electronically. Thus, the etalon 15 will be mounted on a motorized base to enable electronic tuning. This method is slow—potentially longer than a second to tune across the entire band—but is known to be reliable and easy to implement.

In addition to tuning and linewidth narrowing, it is necessary to select the wavelength band (around 1570 nm, 1617 nm, or 1645 nm). The free-running wavelength of low-doped Er:YAG is 1645 nm; if no spectral filtering is included, the laser will run at this wavelength. The etalon is useful to narrow the linewidth, but not to select the wider wavelength range; other spectral filters must be used. For this reason, the switchable spectral filter 16 will be a filter wheel with three openings. One opening is blank; this is for operation at 1645 nm. The other two have spectral selection filters.

A number of spectral filters were considered for selecting the alternate lines. The apparatus may be “reconfigurable” to operate at the other wavelengths, so there could be a requirement for mirror exchange to change the wavelength range. Adding spectral filtering to the cavity can be accomplished without the need to open the cavity itself. The spectral filters, then, may be bandpass or shortpass; shortpass will work because the 1617 nm line has a higher emission cross-section than the 1570 nm lines.

Four methods of spectral filtering were considered, two bandpass and two shortpass. For bandpass volume Bragg gratings and interference filters; for shortpass, atomic or molecular filters and interference filters. Of all these, the items with the lowest insertion loss are the shortpass interference filters. The center wavelengths of the shortpass filters should be 1630 nm and 1590 nm. When no filter is used, the laser will operate at 1645 nm. When the wheel is rotated and the 1630 nm shortpass filter is in place, the additional loss at 1645 nm is 90% while the insertion loss at 1617 nm is <1% (it is about the same at 1570 nm), and the 1617 nm line will oscillate. When the 1590 nm shortpass filter is in place, the loss at 1645 nm is 95% and the loss at 1617 nm is 92%, but the insertion loss at 1570 nm is <1%. Thus, with this filter rotated into place, the subject invention will output 1570 nm.

To create short pulses, laser systems usually rely on Q-switching. In this mode, the laser is pumped continually, but the losses are kept high—so the cavity lifetime (eq. (0-4)) is short. This prevents a buildup of the photon flux, φ, in the cavity, forcing the inversion to get very large. When the cavity quality is switched into much lower loss, the large inversion converts into large photon flux, producing a pulse that contains most of the energy stored in the inversion during the high-loss time period. This can generate pulses whose lengths are between 20 ns and 100 ns, and whose peak power is thousands or millions of times higher than the same laser running in CW mode.

There are two difficulties with traditional Q-switching. First, the optimal repetition rate for a Q-switched laser is less than the inverse of the upper laser level lifetime, which would limit the Er:YAG laser to ˜100 Hz repetition rate (more than two orders of magnitude less than the required value of 20 kHz). Above this, the pulses become longer and lower power. Second, the cross-relaxation inherent in Er:YAG depopulates the upper laser level at a rate proportional to the square of the population in that level—and Q-switching depends on populating that level.

Operation of the switch involves a nonlinear optical crystal and a polarizer, such as β-barium borate (BaB₂O₄, or BBO). When properly cut, this crystal rotates the polarization of light passing through it, and rotates the polarization differently depending on its direction of polarization. By adjusting the voltage on the crystal, the polarization can be switched to pass light without loss, or to completely switch the light out of the cavity (FIGS. 4(a) and (b).

In half-wave mode (FIG. 4(a), light that is vertically polarized passes through the polarizing beamsplitter 21. As it passes through the nonlinear crystal 20, the polarization is rotated 90°, resulting in horizontal linear polarization. After reflection from mirror 22 it passes through the crystal again, rotating the polarization another 90°, and it is vertically polarized again, so it passes back through the beamsplitter without loss. In quarter-wave mode (FIG. 4(b), however, the relative polarization rotation results in conversion of vertical linear polarization to clockwise circular polarization, which becomes counterclockwise upon reflection. This is rotated again upon passage through the crystal, and is horizontal when it reaches the beamsplitter. The polarization is then switched out of the cavity. By rapidly changing the voltage across the nonlinear crystal, the polarization rotation can be switched from quarter- to half-wave in <1 ns, turning the nonlinear crystal into a high-speed optical switch.

For this apparatus cavity-dumping is used. This is almost the inverse of Q-switching; it depends on storing power in the photon field rather than in the inversion. The laser still builds an inversion, which is then switched into a low-loss mode, but the inversion does not need to be anywhere near as large as in the Q-switched case. The mirrors containing the beam are high reflectivity, so the energy in the inversion is rapidly converted to photon flux. Then the cavity is switched again, and the photon flux is dumped out of the cavity in a short pulse (for the design described in Section 0, we anticipate 3-5 ns pulse width).

The rise time of the pulse depends slightly on internal cavity loss and strongly on switching time. Its fall time depends on total cavity loss, internal+output. The pulse length can be increased by ˜25% without loss of pulse energy, but much longer leads to reduced output.

One potential application of the subject invention is atmospheric monitoring, using dual-wavelength absorption lidar. It is possible, for example, to use two wavelengths near 1570 nm to monitor atmospheric CO₂. In some cases it would be useful to generate two wavelengths more widely separated, such as 1645.13 nm (CH₄ absorption) or 1571.11 nm (CO₂ absorption), and 1617.42 nm (minimum absorption).

There are optimal locations for two laser rods in the laser cavity. For dual wavelength absorption lidar, each of these lasers can be set to its own specific wavelength. Each will have its own set of optics but the wavelength selection can be set to specific values; by judicious mirror design, the switchable spectral filter can be removed entirely. The system can be set to any of the ˜20 available lines in Er:YAG, and the two rods can be set to slightly different wavelengths in the same laser transition or to wavelengths up to 100 nm apart.

Repetition rate can be up to 20 kHz (this is adjustable by slightly reducing the pump rate and decreasing the cavity-dump rate, the repetition rate decreases), with pulse energy >1 mJ. One design includes a rod 5 mm in diameter and 5 cm long, pumped by 1.47-μm laser diodes in the laser cavity. These are the parameters used to calculate the size, weight, power, and cost of the apparatus of the subject invention.

One embodiment is shown in FIG. 1. A removable short-pass filter may be added to demonstrate lasing at various wavelengths. The apparatus in FIG. 1 uses the end-pumped configuration; the HR mirror 14 is dichroic, passing 1.47 μm but high reflectivity at 1.57-1.65 μm. This pumps the laser crystal; for any wavelength except 1.645 μm, a shortpass filter may be added between the HR mirror and the laser crystal. The polarizing beamsplitter will select one polarization (as shown, vertical linear polarization) to be reflected through the BBO crystal and to the other HR mirror, forming a laser cavity.

In free-running or CW operation, the voltage across the BBO crystal will be set to half-wave rotation, so that the photon flux is reflected back between the mirrors; there is still a considerable amount of waste heat. To generate the cavity-dumped pulse, with output towards the bottom of the photo, the voltage across the BBO crystal will be dropped to quarter-wave rotation; after two passes it is horizontally polarized and exits the cavity.

It will be understood that the foregoing description is of preferred exemplary embodiments of the invention and that the invention is not limited to the specific forms shown or described herein. Various modifications may be made in the design, arrangement, and type of elements disclosed herein, as well as the steps of making and using the invention without departing from the scope of the invention as expressed in the appended claims.

1. A remote sensing laser apparatus, comprising:
solid-state laser, including
an Er:YAG laser, side pumped by semiconductor lasers in the Erbium absorption band and having an intracavity etalon and a switchable spectral filter.

2. The laser apparatus of claim 1, wherein the laser is line-selectable to the CO₂ wavelength range and the CH₄ wavelength range.

3. The laser apparatus of claim 1, wherein the laser is pulsed.

4. The laser apparatus of claim 1, wherein the laser is cavity dumped.

5. The laser apparatus of claim 1, wherein the intracavity etalon both narrows linewidth and tunes within a wavelength band.

6. The laser apparatus of claim 3, wherein the pulsed laser has a constant pulse energy.

7. The laser apparatus of claim 3, wherein the pulses are >1 mJ/pulse in <10 ns.

8. A laser apparatus comprising:
an Er:YAG laser;
side pumped by semiconductor lasers;
an intracavity elaton; and
a switchable spectral filter,
where the laser is line-selectable to the CO₂ and CH₄ wavelengths, and
the laser is pulsed and cavity dumped.
